(19) United States
(12) Patent Application Publication
(10) Pub. No.: US 2026/0124355 A1
(43) Pub. Date: May 7, 2026

(54) PUNCTURE LOCK CATCH CONNECTOR AND GLUE-FREE AND CHEMICAL-SOLVENT-FREE INFUSION ASSEMBLY

(71) Applicants: VINCENT MEDICAL ADMIRED CO., LTD., Dongguan (CN); VINCENT MEDICAL (DONG GUAN) TECHNOLOGY CO., LTD., Dongguan (CN); VINCENT MEDICAL (DONG GUAN) MANUFACTURING CO., LTD., Dongguan (CN)

(72) Inventors: Kam Man NG, Dongguan (CN); Siu Tung LI, Dongguan (CN); Zhiwei FENG, Dongguan (CN); Cheung Tai Raymond CHOI, Dongguan (CN)

(73) Assignees: VINCENT MEDICAL ADMIRED CO., LTD., Dongguan (CN); VINCENT MEDICAL (DONG GUAN) TECHNOLOGY CO., LTD., Dongguan (CN); VINCENT MEDICAL (DONG GUAN) MANUFACTURING CO., LTD., Dongguan (CN)

(21) Appl. No.: 19/246,916

(22) Filed: Jun. 24, 2025

(30) Foreign Application Priority Data
Nov. 4, 2024 (CN) 202422687270.6

(51) Int. Cl. A61M 5/14; A61M 39/10

(52) U.S. Cl. CPC A61M 5/1413; A61M 39/1011

(57) ABSTRACT

A puncture lock catch connector and a glue-free and chemical-solvent-free infusion assembly are provided, which relate to the field of medical device technologies. The puncture lock catch connector has the following elements integrally molded and disposed, in sequence, from top to bottom: a tapered puncture head, an oblique puncture hole being formed in the tapered puncture head; a stopper plate, the stopper plate has a puncture end and a holding end that are relatively disposed, where the tapered puncture head is perpendicularly disposed on the puncture end; and the stopper plate is configured to limit a displacement of the tapered puncture head; a holding part, the holding part having a square body, where one end of the square body is disposed perpendicularly to the stopper plate; and a connection tube, where one end of the connection tube is fastened to the other end of the square body.

### CROSS-REFERENCE TO RELATED APPLICATIONS

The application claims priority of Chinese patent application No. 202422687270.6 filed on Nov. 4, 2024, which is incorporated herein by reference in its entireties.

### TECHNICAL FIELD

The present disclosure relates to the field of medical device technologies, and in particular, to a puncture lock catch connector and a glue-free and chemical-solvent-free infusion assembly.

### BACKGROUND

Currently, a puncture head and a holding part are usually disposed in a puncture device body part of an infusion tube assembly.

However, a holding part of an existing puncture device is mostly set as a cylindrical structure, so that in a case of unstable holding that may occur when a medical worker holds a puncture device, slippage is prone to occur. Once holding is unstable, the puncture head may accidentally stab the skin, causing potential safety risks to the medical worker when using the puncture device.

Therefore, defects and deficiencies exist in the prior art, and need to be further improved and developed.

### SUMMARY

In view of the foregoing shortcomings of the prior art, the purpose of the present disclosure is to provide a puncture lock catch connector and a glue-free and chemical-solvent-free infusion assembly, aiming at solving the problem that a puncture device in the prior art is prone to hold unstably to bring potential safety hazards to a medical worker.

A technical solution adopted for solving the technical problem in the present disclosure is as follows. A puncture lock catch connector is configured for infusion of a patient. The puncture lock catch connector has the followings integrally molded and disposed in sequence from top to bottom:
a tapered puncture head, an oblique puncture hole being formed in the tapered puncture head;
a stopper plate, the stopper plate having a puncture end and a holding end that are relatively disposed, where the tapered puncture head is perpendicularly disposed on the puncture end; and the stopper plate is configured to limit a displacement of the tapered puncture head;
a holding part, the holding part having a square body, where one end of the square body is disposed perpendicularly to the stopper plate; and
a connection tube, where one end of the connection tube is fixed to the other end of the square body; and a liquid outlet hole is formed in the other end of the connection tube, and the liquid outlet hole is disposed in communication with the oblique puncture hole.

Optionally, the square body has a first end face and a second end face that are relatively disposed. A first holding wing plate and a second holding wing plate are disposed at a corner of the first end face, and a third holding wing plate and a fourth holding wing plate are disposed at a corner of the second end face.

The first holding wing plate, the second holding wing plate, the third holding wing plate, and the fourth holding wing plate are all disposed perpendicularly to the stopper plate.

Optionally, a first n-shaped groove is formed between the first holding wing plate and the second holding wing plate, a second n-shaped groove is formed between the third holding wing plate and the fourth holding wing plate, a third n-shaped groove is formed between the first holding wing plate and the third holding wing plate, and a fourth n-shaped groove is formed between the second holding wing plate and the fourth holding wing plate.

Optionally, an air inlet tube is disposed in the third n-shaped groove, and the air inlet tube is disposed in communication with the liquid outlet hole and the oblique puncture hole.

Optionally, a plurality of reinforcing members are disposed in the third n-shaped groove and the fourth n-shaped groove.

Optionally, the plurality of reinforcing members are capable of being set as cylindrical plates, rib plates, or diaphragm plates.

Another technical scheme adopted for solving the technical problems in the present disclosure is as follows. A glue-free and chemical-solvent-free infusion assembly includes:
a puncture lock catch connector as mentioned above, where the connection tube includes a connection tube body, a buckle plate, and an inverse buckle member, the buckle plate is annularly disposed on a side, close to the holding part, of the connection tube body, and the inverse buckle member is annularly disposed at an end, away from the holding part, of the connection tube body;
an infusion tube, where the infusion tube is detachably connected to the puncture lock catch connector; and
a lock catch member, where a penetration hole is formed in the lock catch member, the penetration hole is formed in an end, deviating from the connection tube, of the lock catch member, and the penetration hole is used for the infusion tube to penetrate; where
the lock catch member is configured to fasten the infusion tube to the puncture lock latch connector.

Optionally, the lock catch member is set as a twist lock connector, and a plurality of twist lock protrusions are disposed towards an inner wall of the connection tube; and
a plurality of lock catch protrusions disposed in a one-to-one correspondence with the plurality of twist lock protrusions are disposed on the buckle plate, twist lock openings are formed in the plurality of lock catch protrusions, and a size of the twist lock opening is matched with that of the twist lock protrusion; and an anti-loosening protrusion is disposed at an entrance of the twist lock opening, and the anti-loosening protrusion is configured to prevent loosening of the twist lock protrusion that is clamped into the twist lock opening.

The infusion tube penetrates from the penetration hole, the infusion tube is in sleeve connection into the connection tube body. The twist lock protrusion abuts against the buckle plate to rotate the buckle connector. The plurality of twist lock protrusions are screwed into the twist lock openings.

Optionally, the lock catch member is set as an inverse buckle connector, the inverse buckle connector includes a plurality of squeezing members, and the plurality of squeezing members are fixedly disposed on an end, away from the penetration hole, of the inverse buckle connector; and the squeezing member includes a deformation part and a buckle part.

Optionally, an abutting section, a side support section, and a clamping section are integrally molded and disposed on the buckle part.

The infusion tube penetrates from the penetration hole, and the infusion tube is in sleeve connection into the connection tube body, so that the inverse buckle connector is displaced in a direction of the puncture lock catch connector. The abutting section abuts against the buckle plate. The deformation part is splayed out to drive the deformation part to be bent. The side support part abuts against a side wall of the buckle plate. The clamping section is clamped into the buckle plate.

Compared with the prior art, the present disclosure provides a puncture lock catch connector and a glue-free and chemical-solvent-free infusion assembly. The puncture lock catch connector has a square body by using the holding part, and one end of the square body is disposed perpendicularly to the stopper plate. One end of the connection tube is fastened to the other end of the square body. And then, compared with an existing cylindrical holding structure, a frictional force during holding is increased, a sliding problem of a medical worker caused by sweaty hands or improper hand gestures is avoided to ensure higher stability and safety in an operating process. A liquid outlet hole is formed in the other end of the connection tube, and the liquid outlet hole is disposed in communication with the oblique puncture hole. And then, liquid flow is smoother, the infusion efficiency is improved, gas retention and blockage problems are reduced, and smooth infusion is further guaranteed.

### BRIEF DESCRIPTION OF THE DRAWINGS

FIG. 1 is a stereo schematic structural diagram of a puncture lock catch connector provided in the present disclosure.

FIG. 2 is another stereo schematic structural diagram of a puncture lock catch connector provided in the present disclosure.

FIG. 3 is another stereo schematic structural diagram of a puncture lock catch connector provided in the present disclosure.

FIG. 4 is another stereo schematic structural diagram of a puncture lock catch connector provided in the present disclosure.

FIG. 5 is another stereo schematic structural diagram of a puncture lock catch connector provided in the present disclosure.

FIG. 6 is a stereo exploded schematic structural diagram of a glue-free and chemical-solvent-free infusion assembly in the present disclosure.

FIG. 7 is another stereo exploded schematic structural diagram of a glue-free and chemical-solvent-free infusion assembly in the present disclosure.

FIG. 8 is another stereo exploded schematic structural diagram of a glue-free and chemical-solvent-free infusion assembly in the present disclosure.

FIG. 9 is another stereo exploded schematic structural diagram of a glue-free and chemical-solvent-free infusion assembly in the present disclosure.

### DETAILED DESCRIPTION OF THE EMBODIMENTS

Embodiments of the present disclosure are described in detail below, and examples of the embodiments are illustrated in the attached figures, where same or similar mark numbers refer to the same or similar elements or elements with the same or similar function throughout. The embodiments described below by reference to the attached figures are exemplary, and merely used for explaining the present disclosure and are not to be construed as limiting the present disclosure.

In the description of the present disclosure, it needs to be illustrated that the indicative direction or position relations of the terms such as “central”, “longitudinal”, “transverse”, “upper”, “lower”, “front”, “rear”, “left”, “right”, “vertical”, “horizontal”, “top”, “bottom”, “inside” and “outside” are direction or position relations illustrated based on the attached figures, just for facilitating the description of the present disclosure and simplifying the description, but not for indicating or hinting that the indicated device or element must be in a specific direction and is constructed and operated in the specific direction, the terms cannot be understood as the restriction of the present disclosure. In addition, the terms “first” and “second” are merely intended for a purpose of description, and shall not be understood as an indication or implication of relative importance or implicit indication of the number of indicated technical features. Therefore, a feature limited by “first” or “second” may explicitly or implicitly include one or more features. In the descriptions of the present disclosure, unless otherwise stated, “a plurality of” means at least two.

In the description of the present disclosure, it needs to be illustrated that, except as otherwise noted, the terms such as install”, “link” and “connect” should be generally understood, for example, the components can be fixedly connected, and also can be detachably connected or integrally connected; the components can be mechanically connected, and also can be electrically connected; the components can be directly connected and also can be indirectly connected through an intermediate, and two components can be communicated internally. For any person skilled in the art, the specific meanings of the terms in the present disclosure can be understood according to specific conditions.

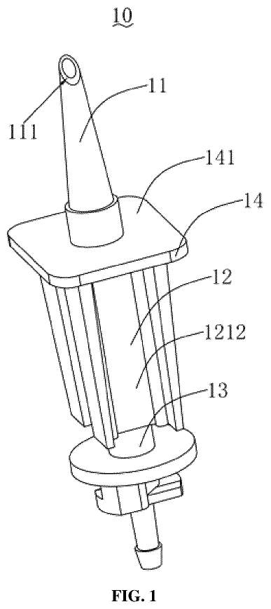

With reference to FIG. 1, a first embodiment of the present disclosure provides a puncture lock catch connector 10 used for infusion of a patient. Firstly, tapered arrangement of a puncture head allows the puncture head to be inserted into a liquid medicine bag or a medicine bottle to form a stable infusion channel. An oblique puncture hole 111 formed in the puncture end ensures that liquid can quickly flow into the infusion channel, thereby reducing a possibility of blockage. Secondly, a holding part 12 is a structure of a square body 121 to provide a better gripping experience. A medical worker controls an angle and a depth of the puncture lock catch connector by using the holding part 12 to ensure stability in a puncture process and reduce an operational risk caused by unstable gripping. Finally, one end of the connection tube 13 is connected to the puncture head, and the other end is connected to an infusion tube 21 by using a liquid outlet hole 131. When the tapered puncture head 11 is inserted into the liquid medicine bag, liquid can smoothly flow into the infusion tube 21, thereby ensuring continuity and high efficiency of an infusion process. In addition, arrangement of a lock catch member 22 ensures the safe connection of the infusion tube 21 during operation, thereby effectively preventing liquid leakage, and improving safety and reliability of overall use.

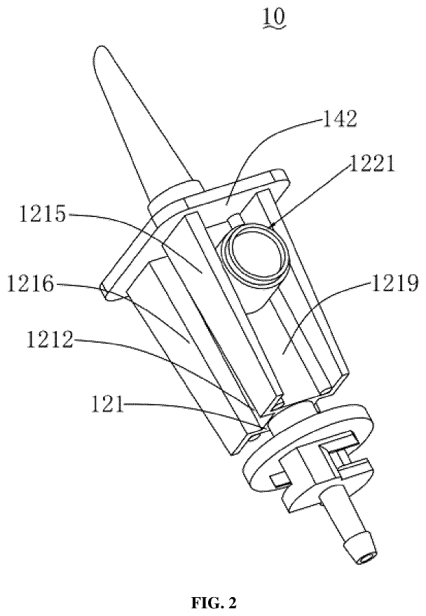

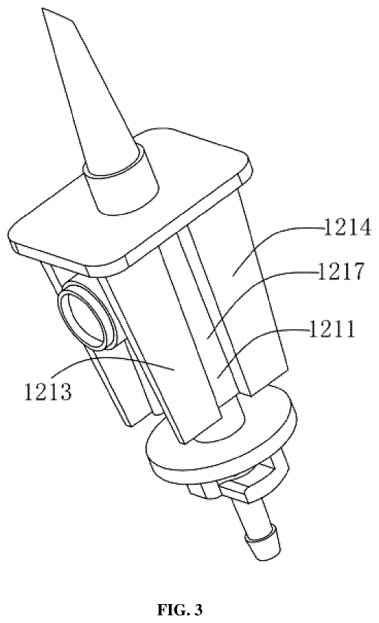

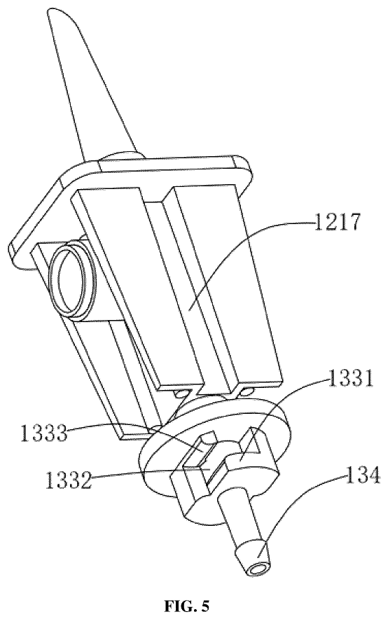

With reference to FIG. 2 to FIG. 5, in some embodiments, the puncture lock catch connector 10 has a tapered puncture head 11, a stopper plate 14, a holding part 12 and a connection tube 13 that are integrally molded and disposed in sequence from top to bottom. An oblique puncture hole 111 is formed in the tapered puncture head 11, and then liquid flows into a path more smoothly, thereby reducing possibility of blockage or liquid reflux. The stopper plate 14 has a puncture end 141 and a holding end 142 that are relatively disposed, and the tapered puncture head 11 is perpendicularly disposed on the puncture end 141. The stopper plate 14 is configured to limit a displacement of the tapered puncture head 11, effectively limit excessive displacement of the tapered puncture head 11 and prevent the tapered puncture head 11 from being punctured too deeply or being shed, thereby improving safety and precision of operation. The holding part 12 has a square body 121, and one end of the square body 121 is disposed perpendicularly to the stopper plate 14. One end of the connection tube 13 is fixed to the other end of the square body 121. And then, compared with an existing cylindrical holding structure, a frictional force during holding is increased, a sliding problem of the medical worker caused by sweaty hands or improper hand gestures is avoided to ensure higher stability and safety in an operating process. A liquid outlet hole 131 is formed in the other end of the connection tube 13, and the liquid outlet hole 131 is disposed in communication with the oblique puncture hole 111. And then, the liquid flows more smoothly, thereby improving infusion efficiency, reducing problems of gas retention and blockage, and further guaranteeing smooth infusion.

With reference to FIG. 2 to FIG. 5, in some embodiments, the square body 121 has a first end face 1211 and a second end face 1212 that are relatively disposed. A first holding wing plate 1213 and a second holding wing plate 1214 are disposed at a corner of the first end face 1211. A third holding wing plate 1215 and a fourth holding wing plate 1216 are disposed at a corner of the second end face 1212. And then, additional support points are provided for fingers, so that the square body 121 is more comfortable and stabler during holding, and a feeling of fatigue during long-time operation is reduced. The first holding wing plate 1213, the second holding wing plate 1214, the third holding wing plate 1215, and the fourth holding wing plate 1216 are all disposed perpendicularly to the stopper plate 14. And then, the square body 121 is stressed more uniformly when being held, thereby avoiding a risk of out-of-control holding or local sliding caused by single-point stress, and facilitating the medical worker to control use of a puncture head better.

With reference to FIG. 2 to FIG. 5, in some embodiments, a first n-shaped groove 1217 is formed between the first holding wing plate 1213 and the second holding wing plate 1214, a second n-shaped groove 1218 is formed between the third holding wing plate 1215 and the fourth holding wing plate 1216, a third n-shaped groove 1219 is formed between the first holding wing plate 1213 and the third holding wing plate 1215, and a fourth n-shaped groove 1220 is formed between the second holding wing plate 1214 and the fourth holding wing plate 1216. Therefore, not only a design of the holding part 12 is optimized, but also a holding part structure 12 is lighter, thereby increasing a finger contact surface, increasing a frictional force, and greatly reducing a possibility of holing gripping during operation.

With reference to FIG. 1 and FIG. 2, in some embodiment, an air inlet tube 1221 is disposed in the third n-shaped groove 1219, and the air inlet tube 1221 is disposed in communication with the liquid outlet hole 131 and the oblique puncture hole 111. Therefore, gas in a pipeline can be effectively discharged, and bubbles can be prevented from appearing during infusion, thereby ensuring continuity and stability of liquid flow, and avoiding a problem of infusion unsmoothness or delay caused by gas obstruction.

In some embodiments, a waterproof and breathable membrane is disposed in the air inlet tube 1221, so that air in the air inlet tube 1221 passes through an air inlet hole in the air inlet tube 1221 and the water-proof and breathable film to circulate, so as to prevent liquid from flowing out of the air inlet tube 1221, thereby maintaining an air pressure balance in liquid extraction or infusion process, ensuring continuity and stability of liquid flow, and avoiding a problem of infusion unsmoothness or delay caused by gas obstruction.

With reference to FIG. 2 to FIG. 5, in some embodiments, a plurality of reinforcing members 1222 are disposed in the third n-shaped groove 1219 and the fourth n-shaped groove 1220, and then a reinforced support structure is provided, so that the n-shaped groove has better stability under pressure, thereby preventing a groove opening from being broken or deformed due to excessive force, and ensuring reliability of puncture operation.

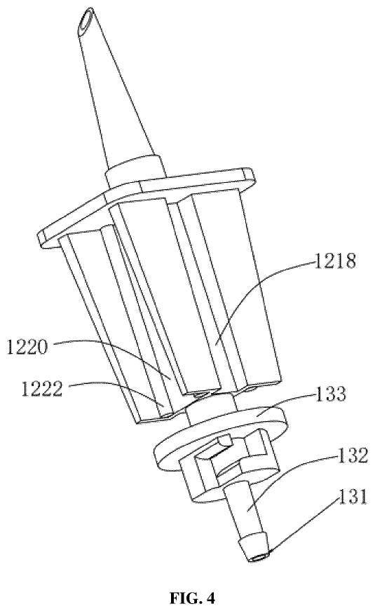

Referring to FIG. 4, in some embodiments, the plurality of reinforcing members 1222 are capable of being set as cylindrical plates, rib plates, or diaphragm plates, and then a structure of the reinforcing member 1222 can be adjusted as required, thereby further optimizing structural strength and compression resistance of the puncture lock catch connector 10, and ensuring reliability of puncture operation of the puncture lock catch connector 10.

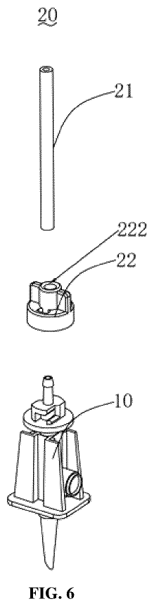

With reference to FIG. 4 and FIG. 6, a second embodiment of the present disclosure provides a glue-free and chemical-solvent-free infusion assembly 20. The glue-free and chemical-solvent-free infusion assembly 20 includes a puncture lock catch connector 10 as mentioned above, an infusion tube 21 and a lock catch member 22. The connection tube 13 includes a connection tube body 132, a buckle plate 133, and an inverse buckle member 134. The buckle plate 133 is annularly disposed on a side, close to the holding part 12, of the connection tube body 132. The inverse buckle member 134 is annularly disposed at an end, away from the holding part 12, of the connection tube body 132. The infusion tube 21 is detachably connected to the puncture lock catch connector 10. A penetration hole 222 is formed in the lock catch member 22. The penetration hole 222 is formed in an end, deviating from the connection tube 13, of the lock catch member 22. The penetration hole 222 is used for the infusion tube 21 to penetrate. The lock catch member 22 is configured to fasten the infusion tube 21 to the puncture lock latch connector 10, thereby greatly improving air seal performance and connection quality between the puncture lock catch connector 10 and the infusion tube 21, avoiding a loosening problem that may be caused by traditional glue connection, abandoning a process of using glue to cure, eliminating a safety risk caused by volatile organic matter volatilization, ensuring safety of a patient, reducing time and cost of producing the glue-free and chemical-solvent-free infusion assembly without air-drying and curing time, and improving production capacity of the glue-free and chemical-solvent-free infusion assembly.

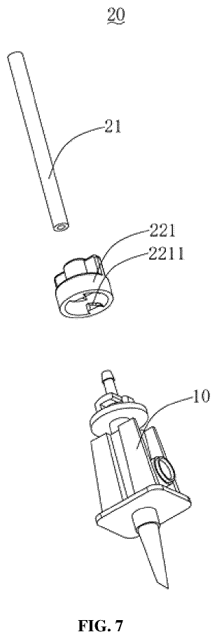

With reference to FIG. 7, in some embodiments, the lock catch member 22 is set as a twist lock connector 221, and a plurality of twist lock protrusions 2221 are disposed towards an inner wall of the connection tube 13. A plurality of lock catch protrusions 1331 disposed in a one-to-one correspondence with the plurality of twist lock protrusions 2211 are disposed on the buckle plate 133. Twist lock openings 1332 are formed in the plurality of lock catch protrusions 133. A size of the twist lock opening 1332 is matched with that of the twist lock protrusion 2211. And then, firm connection with the twist lock opening 1332 on the buckle plate 133 is implemented by using the twist lock protrusion 2211, thereby avoiding a phenomenon of leakage or shedding caused by loosened connection of the infusion tube 21 during infusion, and ensuring continuity and stability of infusion. An anti-loosening protrusion 1333 is disposed at an entrance of the twist lock opening 1332, and the anti-loosening protrusion 1333 is configured to prevent loosening of the twist lock protrusion 2211 that is clamped into the twist lock opening 1332. The infusion tube 21 penetrates from the penetration hole 222. The infusion tube 21 is in sleeve connection into the connection tube body 132. The twist lock protrusion 2211 abuts against the buckle plate 133 to rotate the buckle connector 221. The plurality of twist lock protrusions 2211 are screwed into the twist lock openings 1332. And then, a problem of loosening of the twist lock protrusion 2211 caused by twisting or pulling of the infusion tube 21 during a long period of infusion is prevented, thereby improving overall use safety.

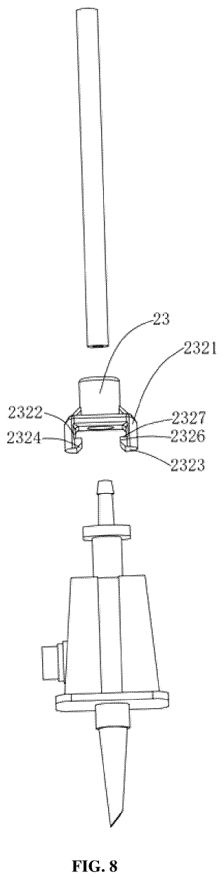

With reference to FIG. 8, in some embodiments, the lock catch member 22 is set as an inverse buckle connector 23. The inverse buckle connector 23 includes a plurality of squeezing members 2321. The plurality of squeezing members 2321 are fixedly disposed on an end, away from the penetration hole 222, of the inverse buckle connector 23. The squeezing member 2321 includes a deformation part 2322 and a buckle part 2323. And then, the inverse buckle connector 23 can be rapidly assembled in an assembly process, and fastness and reliability of connection are greatly improved.

With reference to FIG. 8, in some embodiments, an abutting section 2324, a side support section 2326, and a clamping section 2327 are integrally molded and disposed on the buckle part 2323. The infusion tube 21 penetrates from the penetration hole 222, and the infusion tube is in sleeve connection into the connection tube body 132, so that the inverse buckle connector 23 is displaced in a direction of the puncture lock catch connector 10. The abutting section 2324 abuts against the buckle plate 133. The deformation part 2322 is splayed out to drive the deformation part 2322 to be bent. The side support part 2326 abuts against a side wall of the buckle plate 133. The clamping section 2327 is clamped into the buckle plate 133. And then, by using a combined action of the abutting section 2324, the side support section 2326, and the clamping section 2327, stable and unloosened connection between the infusion tube 21 and the puncture lock catch connector 10 is ensured, and a risk of liquid leakage or device shedding during infusion is further avoided.

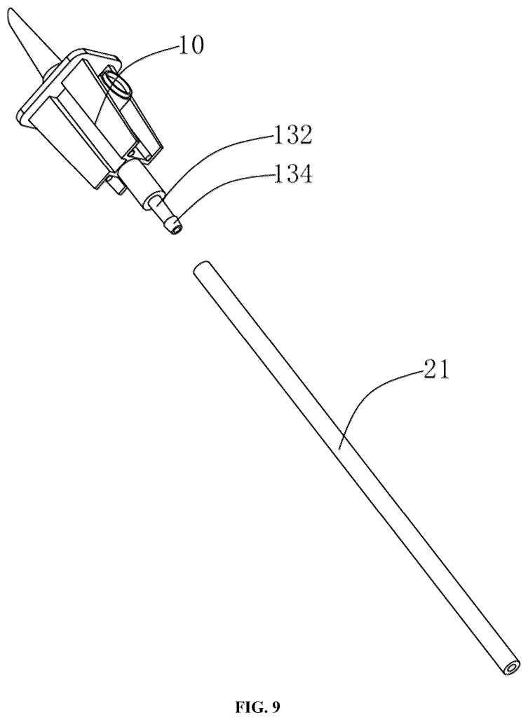

With reference to FIG. 9, in some embodiments, after the infusion tube 21 is directly inserted into the inverse buckle member 134, the infusion tube 21 can be sleeved in the connection tube body 132 to realize an interference fit between the infusion tube 21 and the connection tube 13 body, thereby ensuring stable and unloosened connection between the infusion tube 21 and the puncture lock catch connector 10, and further avoiding a risk of liquid leakage or device shedding during infusion.

In conclusion, the present disclosure provides a puncture lock catch connector and a glue-free and chemical-solvent-free infusion assembly. The puncture lock catch connector has the followings integrally formed and disposed in sequence from top to bottom: a tapered puncture head, an oblique puncture hole being formed in the tapered puncture head; a stopper plate, the stopper plate has a puncture end and a holding end that are relatively disposed, where the tapered puncture head is perpendicularly disposed on the puncture end; and the stopper plate is configured to limit a displacement of the tapered puncture head; a holding part, the holding part having a square body, where one end of the square body is disposed perpendicularly to the stopper plate; and a connection tube, where one end of the connection tube is fastened to the other end of the square body; and a liquid outlet hole is formed in the other end of the connection tube, and the liquid outlet hole is disposed in communication with the oblique puncture hole. And then, compared with an existing cylindrical holding structure, a frictional force during holding is increased, a sliding problem of a medical worker caused by sweaty hands or improper hand gestures is avoided to ensure higher stability and safety in an operating process. A liquid outlet hole is formed in the other end of the connection tube, and the liquid outlet hole is disposed in communication with the oblique puncture hole. And then, liquid flow is smoother, the infusion efficiency is improved, gas retention and blockage problems are reduced, and smooth infusion is further guaranteed.

It should be understood that the application of the present disclosure is not limited to the examples described above, and these modifications or variations can be made according to the above description for those skilled in the art, all of which are intended to fall within the scope of the appended claims.

What is claimed is:

1. A puncture lock catch connector, configured for infusion of a patient, wherein the puncture lock catch connector comprises the following, which are integrally molded and disposed in sequence from top to bottom:
a tapered puncture head, an oblique puncture hole being formed in the tapered puncture head;
a stopper plate, the stopper plate having a puncture end and a holding end that are relatively disposed, wherein the tapered puncture head is perpendicularly disposed on the puncture end; and the stopper plate is configured to limit a displacement of the tapered puncture head;
a holding part, the holding part having a square body, wherein one end of the square body is disposed perpendicularly to the stopper plate; and
a connection tube, wherein one end of the connection tube is fixed to the other end of the square body; and a liquid outlet hole is formed in the other end of the connection tube, and the liquid outlet hole is disposed in communication with the oblique puncture hole.

2. The puncture lock catch connector according to claim 1, wherein the square body has a first end face and a second end face that are relatively disposed, a first holding wing plate and a second holding wing plate are disposed at a corner of the first end face, and a third holding wing plate and a fourth holding wing plate are disposed at a corner of the second end face; and
the first holding wing plate, the second holding wing plate, the third holding wing plate, and the fourth holding wing plate are all disposed perpendicularly to the stopper plate.

3. The puncture lock connector according to claim 2, wherein a first n-shaped groove is formed between the first holding wing plate and the second holding wing plate, a second n-shaped groove is formed between the third holding wing plate and the fourth holding wing plate, a third n-shaped groove is formed between the first holding wing plate and the third holding wing plate, and a fourth n-shaped groove is formed between the second holding wing plate and the fourth holding wing plate.

4. The puncture lock catch connector according to claim 3, wherein an air inlet tube is disposed in the third n-shaped groove, and the air inlet tube is disposed in communication with the liquid outlet hole and the oblique puncture hole.

5. The puncture lock catch connector according to claim 3, wherein a plurality of reinforcing members are disposed in the third n-shaped groove and the fourth n-shaped groove.

6. The puncture lock catch connector according to claim 5, wherein the plurality of reinforcing members are configured to set as cylindrical plates, rib plates, or diaphragm plates.

7. A glue-free and chemical-solvent-free infusion assembly, comprising:
the puncture lock catch connector according to claim 1, wherein the connection tube comprises a connection tube body, a buckle plate, and an inverse buckle member, the buckle plate is annularly disposed on a side, close to the holding part, of the connection tube body, and the inverse buckle member is annularly disposed at an end, away from the holding part, of the connection tube body;
an infusion tube, wherein the infusion tube is detachably connected to the puncture lock catch connector; and
a lock catch member, wherein a penetration hole is formed in the lock catch member, the penetration hole is formed in an end, deviating from the connection tube, of the lock catch member, and the penetration hole is used for the infusion tube to penetrate; wherein the lock catch member is configured to fasten the infusion tube to the puncture lock latch connector.

8. The glue-free and chemical-solvent-free infusion assembly according to claim 7, wherein the lock catch member is set as a twist lock connector, and a plurality of twist lock protrusions are disposed towards an inner wall of the connection tube; and
a plurality of lock catch protrusions disposed in a one-to-one correspondence with the plurality of twist lock protrusions are disposed on the buckle plate, twist lock openings are formed in the plurality of lock catch protrusions, and a size of the twist lock opening is matched with that of the twist lock protrusion; and an anti-loosening protrusion is disposed at an entrance of the twist lock opening, and the anti-loosening protrusion is configured to prevent loosening of the twist lock protrusion that is clamped into the twist lock opening; wherein
the infusion tube penetrates from the penetration hole, the infusion tube is in sleeve connection into the connection tube body, the twist lock protrusion abuts against the buckle plate to rotate the buckle connector, and the plurality of twist lock protrusions are screwed into the twist lock openings.

9. The glue-free and chemical-solvent-free infusion assembly according to claim 7, wherein the lock catch member is set as an inverse buckle connector, the inverse buckle connector comprises a plurality of squeezing members, and the plurality of squeezing members are fixedly disposed on an end, away from the penetration hole, of the inverse buckle connector; and the squeezing member comprises a deformation part and a buckle part.

10. The glue-free and chemical-solvent-free infusion assembly according to claim 9, wherein an abutting section, a side support section, and a clamping section are integrally molded and disposed on the buckle part; wherein
the infusion tube penetrates from the penetration hole, and the infusion tube is in sleeve connection into the connection tube body, so that the inverse buckle connector is displaced in a direction of the puncture lock catch connector, the abutting section abuts against the buckle plate, the deformation part is splayed out to drive the deformation part to be bent, the side support part abuts against a side wall of the buckle plate, and the clamping section is clamped into the buckle plate.
